SEQUENCE LISTING (2 sequences)

SEQ ID NO: 1 - DNA, 1452 bp, Unknown
  Description of Unknown Organism Unknown bacterial strain E-396
agtttgatcctggctcagaacgaacgctggcggcaggcttaacacatgcaagtcgagcgagaccttcgggtctagcggcggacgggtgagtaacgcgtgggaacgtgcccttctctacggaatagccccgggaaactgggagtaataccgtatacgccctttgggggaaagatttatcggagaaggatcggcccgcgttggattaggtagttggtggggtaatggcccaccaagccgacgatccatagctggtttgagaggatgatcagccacactgggactgagacacggcccagactcctacgggaggcagcagtggggaatcttagacaatgggggcaaccctgatctagccatgccgcgtgagtgatgaaggccttagggttgtaaagctctttcagctgggaagataatgacggtaccagcagaagaagccccggctaactccgtgccagcagccgcggtaatacggagggggctagcgttgttcggaattactgggcgtaaagcgcacgtaggcggactggaaagtcagaggtgaaatcccagggctcaaccttggaactgcctttgaaactatcagtctggagttcgagagaggtgagtggaattccgagtgtagaggtgaaattcgtagatattcggaggaacaccagtggcgaaggcggctcactggctcgatactgacgctgaggtgcgaaagcgtggggagcaaacaggattagataccctggtagtccacgccgtaaacgatgaatgccagacgtcggcaagcatgcttgtcggtgtcacacctaacggattaagcattccgcctggggagtacggtcgcaagattaaaactcaaaggaattgacgggggcccgcacaagcggtggagcatgtggtttaattcgaagcaacgcgcagaaccttaccaacccttgacatggcaggaccgctggagagattcagctttctcgtaagagacctgcacacaggtgctgcatggctgtcgtcagctcgtgtcgtgagatgttcggttaagtccggcaacgagcgcaacccacgtccctagttgccagcaattcagttgggaactctatggaaactgccgatgataagtcggaggaaggtgtggatgacgtcaagtcctcatgggccttacgggttgggctacacacgtgctacaatggtggtgacagtgggttaatccccaaaagccatctcagttcggattgtcctctgcaactcgagggcatgaagttggaatcgctagtaatcgcggaacagcatgccgcggtgaatacgttcccgggccttgtacacaccgcccgtcacaccatgggagttggttctacccgacgacgntgcgctaaccttcggggggcaggcggccacggtaggatcagcgactggggtgaagtcgtaacaaggtagccgtaggggaacctgcggctggatcacctcctt

SEQ ID NO: 2 - DNA, 1426 bp, Unknown
  Description of Unknown Organism Unknown bacterial strain A-581-1
tagagtttgatcctggctcagaacgaacgctggcggcaggcttaacacatgcaagtcgagcgagaccttcgggtctagcggcggacgggtgagtaacgcgtgggaacgtgcccttctctacggaatagccccgggaaactgggagtaataccgtatacgccctttgggggaaagatttatcggagaaggatcggcccgcgttggattaggtagttggtgaggtaacggctcaccaagccgacgatccatagctggtttgagaggatgatcagccacactgggactgagacacggcccagactcctacgggaggcagcagtggggaatcttagacaatgggggcaaccctgatctagccatgccgcgtgagtgatgaaggccttagggttgtaaagctctttcagctgggaagataatgacggtaccagcagaagaagccccggctaactccgtgccagcagccgcggtaatacggagggggctagcgttgttcggaattactgggcgtaaagcgcacgtaggcggactggaaagtcagaggtgaaatcccagggctcaaccttggaactgcctttgaaactatcagtctggagttcgagagaggtgagtggaattccgagtgtagaggtgaaattcgtagatattcggaggaacaccagtggcgaaggcggctcactggctcgatactgacgctgaggtgcgaaagcgtggggagcaaacaggattagataccctggtagtccacgccgtaaacgatgaatgccagacgtcggcaagcatgcttgtcggtgtcacacctaacggattaagcattccgcctggggagtacggtcgcaagattaaaactcaaaggaattgacgggggcccgcacaagcggtggagcatgtggtttaattcgaagcaacgcgcagaaccttaccaacccttgacatggcaggaccgctggagagattcagctttctcgtaagagacctgcacacaggtgctgcatggctgtcgtcagctcgtgtcgtgagatgttcggttaagtccggcaacgagcgcaacccacgtccctagttgccagcattcagttgggcactctatggaaactgccggtgataagccggaggaaggtgtggatgacgtcaagtcctcatggcccttacgggttgggctacacacgtgctacaatggtggtgacagtgggttaatccccaaaagccatctcagttcggattgtcctctgcaactcgagggcatgaagttggaatcgctagtaatcgcggaacagcatgccgcggtgaatacgttcccgggccttgtacacaccgcccgtcacaccatgggagttggttctacccgacgacgctgcgctaacccttcggggaggcaggcggccacggtaggatcagcgactggggtgaagtcgtaacaaggtagcca

(19) United States
(12) Patent Application Publication
(10) Pub. No.: US 2006/0121556 A1
(43) Pub. Date: Jun. 8, 2006

(54) Process for producing canthaxanthin

(75) Inventors: Kazuaki Hirasawa, Kanagawa (JP); Akira Tsubokura, Kanagawa (JP); Haruyoshi Mizuta, Kanagawa (JP)

Correspondence Address: HELLER EHRMAN WHITE & MCAULIFFE LLP, 1717 RHODE ISLAND AVE, NW, WASHINGTON, DC, 20036-3001, US

(73) Assignee: NIPPON OIL CORPORATION, TOKYO (JP)

(21) Appl. No.: 10/511,345

(22) PCT Filed: Apr. 7, 2003
(86) PCT No.: PCT/JP03/04398

§ 371 Date: Oct. 15, 2004

(30) Foreign Application Priority Data
Apr. 15, 2002 (JP) 2002-112240

(51) Int. Cl. C12P 23/00; C12N 1/21

(52) U.S. Cl. 435/67; 435/252.3

(57) ABSTRACT

This invention provides a method for producing cantlhaxanithin, which is inexpensive, stably provided, and highly safe. This method comprises steps of: inducing mutation in astaxanthin-producing microorganisms in which the nucleotide sequence of DNA corresponding to its 16S ribosomal RNA is substantially homologous to the nucleotide sequence as shown in SEQ ID NO: 1; obtaining canthaxanthin-producing microorganisms by selecting a mutant having a higher ratio of canthaxanthin produced (% by mass) relative to the total amount of carotenoid produced than that of a parent strain; and recovering canthaxanthin or a carotenoid mixture comprising canthaxanthin from the culture product of the canthaxanthin-producing microorganisms.

### TECHNICAL FIELD

The present invention relates to a method for microbiologically producing canthaxanthin or a carotenoid mixture comprising canthaxanthin, which is useful as a natural red pigment for feed additives, food additives, etc.

### BACKGROUND ART

As a method for improving the color tone of egg yolk, flesh, and skin of poultry such as chickens, addition of canthaxanthin to animal feeds are extensively employed in the world. Canthaxanthin is also used in the animal feed industry to improve the color tone of flesh or skin of seafood such as salmon, trout, red sea bream, or shrimp, and also used in the food industry as a coloring agent for foods and beverages.

It has been known that canthaxanthin is contained in certain species of mushrooms (Botanical Gazette, 112, 228-232, 1950), fish, and crustaceans. Examples of known canthaxanthin-producing microorganisms are those belonging to the genus Brevibacterium (Applied and Environmental Microbiology, 55 (10), 2505, 1989), those belonging to the genus Rhodococcus (JP Patent Publication (Unexamined Application) No. 2-138996), those belonging to the genus Corynebacterium (JP Patent Publication (Unexamined Application) No. 6-343482), and the bacterial strain E-396 belonging to a novel genus (JP Patent Publication (Unexamined Application) No. 2001-95500). As methods of chemical synthesis, oxidation of β-carotene (J. Amer. Chem. Soc., 78, 1427, 1956) and synthesis from 3-oxo-C15 phosplhonium salt (Pure Appl. Chem., 51, 875, 1979) are known.

### DISCLOSURE OF INVENTION

In the aforementioned method for chemically synthesizing canthaxanithin, however, an organic solvent is used. Thus, this method is problematic in terms of safety and the preferences for natural products these days. Conventional culture using microorganisms also has problems in that productivity is low and extraction from natural products is cost-intensive.

Regarding the E-396 strain that is known as a carotenoid compound-producing microorganism, its safety has been already assured and a method for producing highly concentrated carotenoid compounds comprising astaxanthin has been already reported. However, the ratio of canthaxanthin to total carotenoid produced is low.

As an alternative to canthaxanthin, capsanthin that is extracted from a paprika plant may be used to improve the color tone of chicken egg yolk. Since capsanthin is very unstable in terms of heat, light, or the like, and growth of paprika is significantly influenced by weather, it is difficult to provide capsanthin at industrially stable levels.

Accordingly, a method for producing canthaxanthin, which is highly safe and stably provided at a low price, is desired.

As a result of intensive and extensive studies aimed at the solution of the aforementioned problems, the present inventors have found that microorganisms in which the ratio of canthaxanthin is high relative to the total amount of carotenoid produced could be easily obtained by mutating astaxanthin-producing microorganisms. This led to the completion of the present invention.

Specifically, the present invention provides the following means.

(1) A method for producing canthaxanthin comprising steps of: inducing mutation in astaxanthin-producing microorganisms in which the nucleotide sequence of DNA corresponding to its 16S ribosomal RNA is substantially homologous to the nucleotide sequence as shown in SEQ ID NO: 1; obtaining canthaxanthin-producing microorganisms by selecting a mutant having a higher ratio of canthaxanthin produced (% by mass) relative to the amount of carotenoid produced than that of a parent strain; and recovering canthaxanthin or a carotenoid mixture comprising canthaxanthin from the culture product of the canthaxanthin-producing microorganisms.

(2) The method according to (1) above, wherein the ratio of canthaxanthin produced from the canthaxanthin-producing microorganisms is at least 40% by mass relative to the total amount of carotenoid produced.

(3) The method according to (1) above, wherein each of the ratios of β-cryptoxanthin, zeaxanthin, 3-hydroxyechinenone, asteroidenone, adonirubin, adonixanthin, and astaxanthin produced from the canthaxanthin-producing microorganisms is less than 20% by mass relative to the total amount of carotenoid produced.

(4) The method according to any one of (1) to (3) above, wherein the astaxanthin-producing microorganisms are selected from the E-396 strain (which has been deposited at the National Institute of Bioscience and Human-Technology (the International Patent Organism Depositary of the National Institute of Advanced Industrial Science and Technology; AIST Tsukuba Central 6, 1-1-1 Higashi, Tsukuba, Ibaraki, Japan) as of Apr. 27, 1993, under the accession number of FERM BP-4283) and a mutant thereof, and the A-581-1 strain (which has been deposited at the National Institute of Bioscience and Human-Technology (the International Patent Organism Depositary of the National Institute of Advanced Industrial Science and Technology; AIST Tsukuba Central 6, 1-1-1 Higashi, Tsukuba, Ibaraki, Japan) as of May 20, 1994, under the accession number of FERM BP-4671) and a mutant thereof.

The present invention is hereafter described in more detail.

In the method according to the present invention, an astaxanthin-producing microorganism is used as a parent strain for mutation. An example of such a microorganism is an astaxanthin-producing microorganism in which the nucleotide sequence of DNA corresponding to its 16S ribosomal RNA is substantially homologous to the nucleotide sequence as shown in SEQ ID NO: 1. The term “substantially homologous” used herein refers to homology of 98% or higher in view of, for example, error frequency in nucleotide sequencing of DNA.

Specific examples of astaxanthin-producing microorganisms having a sequence substantially homologous to the above sequence include the E-396 strain (FERM BP-4283), the A-581-1 strain (FERM BP-4671), various mutants obtained by mutating and improving the E-396 or A-581-1 strain, and related species thereof. The nucleotide sequence of DNA as shown in SEQ ID NO: 1 corresponds to ribosomal RNA of the E-396 strain, and the nucleotide sequence of DNA as shown in SEQ ID NO: 2 corresponds to ribosomal RNA of the A-581-1 strain. Homology of nucleotide sequences of 16S ribosomal RNA between the E-396 strain and the A-581-1 strain is 99.4%, and this indicates that they are closely related strains. Accordingly, these strains form a group of carotenoid-producing bacteria. In the method according to the present invention, a parent strain used in mutation is defined as an astaxanthin-producing microorganism in which the nucleotide sequence of DNA corresponding to its 16S ribosomal RNA is 98% or more homologous to the nucleotide sequence as shown in SEQ ID NO: 1, i.e., the E-396 strain, the A-581-1 strain, mutants of the E-396 or the A-581-1 strain, and related species thereof.

The E-396 strain, which is exemplified as the astaxanthin-pr-oducing microorganism used in the present invention, is described. This strain was newly isolated by the present inventors and deposited at the National Institute of Bioscience and Human-Technology (the International Patent Organism Depositary of the National Institute of Advanced Industrial Science and Technology) as of Apr. 27, 1993, under the accession number of FERM BP-4283. A more specific example of another microorganism is the A-581-1 strain. This strain was newly isolated by the present inventors and deposited at the National Institute of Bioscience and Human-Technology (the International Patent Organism Depositary of the National Institute of Advanced Industrial Science and Technology) as of May 20, 1994, under the accession number of FERM BP-4671.

In the present invention, the method for mutating astaxanthin-producing microorganisms is not particularly limited as long as the method can induce mutation. Examples of methods which can be used include: chemical methods using mutating agents such as N-methyl-N′-nitro-N-nitrosoguanidine (NTG) or ethyl methanesulfonate (EMS); physical methods such as ultraviolet irradiation or X-ray irradiation; and biological methods using gene recombination or transposon. This mutation may be carried out once. Alternatively, mutation may be carried out twice or more by, for example, preparing a mutant of the astaxanthin-producing microorganism by this mutation and further mutating the resulting mutant.

In the present invention, a mutant in which the ratio of canthaxanthin produced is particularly high relative to the total amount of carotenoid can be selected from among mutants obtained by mutating astaxanthin-producing microorganisms by analyzing the carotenoid compound in the culture solution of the mutant.

For example, this culture method is carried out in the following manner. Specifically, culture is conducted in a medium containing a component that is necessary for the growth of the canthaxanthin-producing microorganisms and that generates a carotenoid compound. Culture method may be shake culture using a test tube, flask, or the like, or via aeration agitation culture. A carotenoid compound may be analyzed by any method as long as the carotenoid compound can be separated and detected thereby. For example, high-performance liquid chromatography, thin-layer chromatography, or paper chromatography can be employed.

In the present invention, canthaxanthin-producing microorganisms are obtained by selecting a mutant in which the ratio of canthaxanthin is high relative to the total amount of carotenoid. The term “total amount of carotenoid” used herein refers to the total amount of carotenoid compounds such as astaxanthin, canthaxanthin, adonixanthin, β-carotene, echinenone, zeaxanthin, β-cryptoxanthin, 3-hydroxyechinenone, asteroidenone, or adonirubin.

The astaxanthin-producing microorganisms such as the E-396 strain simultaneously produce various carotenoid compounds such as astaxanthin, canthaxanthin, adonixanthin, β-carotene, echinenone, zeaxanthin, β-cryptoxanthin, 3-hydroxyechinenone, asteroidenone, or adonirubin. Thus, the ratio of canthaxanthin relative to the total amount of carotenoid is low, and it is generally about 2% to 20%.

A mutant that is selected in the present invention induces mutation in astaxanthin-producing microorganisms and the ratio of canthaxanthin produced is particularly high relative to the total amount of carotenoid. The minimum condition for the ratio of canthaxanthin employed as a standard for selection is that this ratio is higher than that of the parent strain before mutation for producing canthaxanthin. A mutant is selected that produces carotenoid containing preferably at least 40% canthaxanthin by mass, and more preferably at least 60% by mass, based on the total amount of carotenoid produced.

The biosynthesis of astaxanthin is deduced as follows. The 6-membered rings at both terminuses of β-carotene are modified by ketolase and hydroxylase. If the deletion of this hydroxylase is complete, it is deduced that only β-carotene, echinenone, and canthaxanthin are produced, and that β-cryptoxanthin, zeaxanthin, 3-hydroxyechinenone, asteroidenone, adonirubin, adonixanthin, and astaxanthin, which are in need of hydroxylase, are not produced. If the deletion of this hydroxylase is incomplete, it is deduced that the ratios of β-cryptoxanthin, zeaxanthin, 3-hydroxyechinenone, asteroidenone, adonirubin, adonixanthin, and astaxanthin relative to the total amount of carotenoid are lowered. Accordingly, as another effective means to select canthaxanthin-producing microorganisms from among mutants, selection can be made based on the phenomenon that the ratios of β-cryptoxanthin, zeaxanthin, 3-hydroxyechinenone, asteroidenone, adonirubin, adonixanthin, and astaxanthin relative to the total amount of carotenoid are low. Selection can be made based on the phenomenon that the ratio of each compound relative to the total carotenoid is preferably less than 20% by mass, and more preferably less than 10% by mass.

In the present invention, the method for culturing canthaxanthin-producing microorganisms to recover canthaxanthin or a carotenoid mixture comprising canthaxanthin may be any method as long as canthaxanthin is produced. Examples of methods that can be used are as follows. Specifically, a medium comprises carbon sources, nitrogen sources, inorganic salts, and if necessary particular nutritional requirements (e.g., vitamins, amino acids, or nucleic acids) that are necessary in the growth of canthaxanthin-producing microorganisms. Examples of carbon sources include: saccharides such as glucose, sucrose, fructose, trehalose, mannose, mannitol, and maltose; organic acids such as acetic acid, fumaric acid, citric acid, propionic acid, malic acid, and malonic acid; and alcohols such as ethanol, propanol, butanol, pentanol, hexanol, and isobutanol. The amount of carbon sources added varies depending their types, and it is generally 1 to 100 g, preferably 2 to 50 g per liter of medium. Examples of nitrogen sources include potassium nitrate, ammonium nitrate, ammonium chloride, ammonium sulfate, ammonium phosphate, ammonia, and urea. These may be used alone or in combination of two or more. The amount of nitrogen sources added varies depending on their types, and it is generally 0.1 to 20 g, preferably 1 to 10 g per liter of medium. Examples of inorganic salts include monobasic potassium phosphate, dibasic potassium phosphate, disodium hydrogen-phosphate, magnesium sulfate, magnesium chloride, iron sulfate, iron chloride, manganese sulfate, manganese chloride, zinc sulfate, zinc chloride, copper sulfate, calcium chloride, calcium carbonate, and sodium carbonate. These may be used alone or in combination of two or more. The amount of inorganic salts added varies depending on their types, and it is generally 0.1 mg to 10 g per liter of medium. Examples of particular nutritional requirements include vitamins, nucleic acids, yeast extract, peptone, meat extract, malt extract, corn steep liquor, dry yeast, soybean cake, soybean oil, olive oil, corn oil, and linseed oil. These may be used alone or in combination of two or more. The amounts of particular nutritional requirements added vary depending on their types, and it is generally 0.01 mg to 100 g per liter of medium. The pH of the medium is adjusted to between 2 and 12, preferably between 6 and 9. Shake culture or aeration agitation culture is carried out at 10° C. to 70° C., preferably 20° C. to 35° C. generally for 1 to 20 days, and preferably for 2 to 9 days.

Subsequently, moisture is removed from the thus obtained culture solution. The amount of moisture, which should be removed from the culture solution in order to obtain a canthaxanthin-containing substance, varies depending on conditions such as a pigment content of the culture solution. In general, filtration is first carried out, and a precipitate is dehydrated if moisture should be further removed. Filtration can be carried out by commonly employed methods such as filtration or centrifugation. When the content of carotenoid compound in the precipitate should be increased, moisture can be removed by dehydrating the precipitate. Examples of dehydration methods include general spray drying, drum-drying, and freeze-drying.

The thus obtained culture precipitate of canthaxanthin-containing microorganisms can be used in that state as a substance containing a pigment for feed additives. When the canthaxanthin and the carotenoid compound obtained by the method of the present invention are used as a coloring agent for feeds, etc., antioxidants such as butylhydroxytoluene, ethoxyquin, or vitamin E can be added to prevent the canthaxanthin and the carotenoid compound from being degraded. Further, the surfaces thereof may be covered with gelatin, etc.

This specification includes part or all of the contents as disclosed in the specification of Japanese Patent Application No. 2002-112240, which is the base of priority of the present invention.

### BEST MODE FOR CARRYING OUT THE INVENTION

The present invention is hereafter described in more detail with reference to the examples but is not limited thereto.

### EXAMPLE 1

The E-396 strain (FERM BP-4283, the ratio of canthaxanthin produced: 7.4% by mass) was allowed to stand at 28° C. for 30 minutes and subjected to mutation with 150 mg/L NTG (N-methyl-N′-nitro-N-nitrosoguanidine). A medium (6 ml) having a composition as shown in Table 1 was placed in a test tube (inner diameter: 18 mm) and steam-sterilized at 121° C. for 15 minutes to prepare a test tube medium. Each of the 400 mutants that were subjected to colony isolation was inoculated on a test tube medium by means of an inoculating loop, and reciprocal shake culture (300 rpm) was carried out at 28° C. for 4 days. Subsequently, this culture product was centrifuged, and the resultant cells were analyzed for their carotenoid compound by high-performance liquid chromatography. As a result, one strain that exhibited a ratio of canthaxanthin relative to the total amount of carotenoid produced of 60% by mass or higher was obtained. The results of analysis of the carotenoid compound in this strain are shown in Table 2.

TABLE 1

| Composition | Amount added, g/L |
|---|---|
| Yeast extract | 20 |
| Peptone | 5 |
| Sucrose | 50 |
| KH₂PO₄ | 1.5 |
| Na₂HPO₄.12H₂O | 3.8 |
| MgSO₄.7H₂O | 0.5 |
| FeSO₄.7H₂O | 0.01 |
| CaCl₂.2H₂O | 0.01 |
| Na₂CO₃ | The amount with which the pH |
| | of the medium reaches 7 |

TABLE 2

| | Concentration of product | |
|---|---|---|
| | per culture solution | Ratio of product |
| Carotenoid compound | mg/L | % by mass |
| β-Carotene | 0.3 | 7.1 |
| Echinenone | 0.5 | 11.9 |
| 3-Hydroxyechinenone | 0.0 | 0.0 |
| Canthaxanthin | 2.7 | 64.3 |
| Adonirubin | 0.6 | 14.3 |
| β-Cryptoxanthin | 0.0 | 0.0 |
| Astaxanthin | 0.1 | 2.4 |
| Asteroidenone | 0.0 | 0.0 |
| Adonixanthin | 0.0 | 0.0 |
| Zeaxanthin | 0.0 | 0.0 |

### EXAMPLE 2

The E-396 strain (FERM BP-4283) was mutated with NTG, colonies with a deep red color were selected, and variant Y-1071 (the ratio of canthaxanthin produced: 6.5% by mass) with enhanced productivity of astaxanthin was obtained. This Y-1071 strain was further mutated with NTG. A medium (6 ml) having a composition as shown in Table 1 was placed in a test tube (inner diameter: 18 mm) and steam-sterilized at 121° C. for 15 minutes to prepare a test tube medium. Each of the 1,000 mutants that were subjected to colony isolation was inoculated on a test tube medium by means of an inoculating loop, and reciprocal shake culture (300 rpm) was carried out at 28° C. for 4 days. Subsequently, this culture product was centrifuged, and the resultant cells were analyzed for their carotenoid compound by high-performance liquid chromatography. As a result, two strains in which each of the ratios of β-cryptoxanthin, zeaxanthin, 3-hydroxyechinenone, asteroidenone, adonirubin, adonixanthin, and astaxanthin relative to the total amount of carotenoid was less than 10% by mass were obtained. The results of analysis of the carotenoid compound in these two strains are shown in Table 3 and Table 4.

TABLE 3

| | Concentration of product | |
|---|---|---|
| | per culture solution | Ratio of product |
| Carotenoid compound | mg/L | % by mass |
| β-Carotene | 0.8 | 8.1 |
| Echinenone | 1.1 | 11.1 |
| 3-Hydroxyechinenone | 0.0 | 0.0 |
| Canthaxanthin | 7.0 | 70.7 |
| Adonirubin | 0.9 | 9.1 |
| β-Cryptoxanthin | 0.0 | 0.0 |
| Astaxanthin | 0.1 | 1.0 |
| Asteroidenone | 0.0 | 0.0 |
| Adonixanthin | 0.0 | 0.0 |
| Zeaxanthin | 0.0 | 0.0 |

TABLE 4

| | Concentration of product | |
|---|---|---|
| | per culture solution | Ratio of product |
| Carotenoid compound | mg/L | % by mass |
| β-Carotene | 0.6 | 5.5 |
| Echinenone | 0.7 | 6.4 |
| 3-Hydroxyechinenone | 0.0 | 0.0 |
| Canthaxanthin | 9.6 | 88.1 |
| Adonirubin | 0.0 | 0.0 |
| β-Cryptoxanthin | 0.0 | 0.0 |
| Astaxanthin | 0.0 | 0.0 |
| Asteroidenone | 0.0 | 0.0 |
| Adonixanthin | 0.0 | 0.0 |
| Zeaxanthin | 0.0 | 0.0 |

### EXAMPLE 3

The A-581-1 strain (FERM4 BP-4671, the ratio of canthaxanthin produced: 5.3% by mass) was mutated by irradiatiing it with ultraviolet light using a UV lamp. A medium (6 ml) having a composition as shown in Table 1 was placed in a test tube (inner diameter: 18 mm) and steam-sterilized at 121° C. for 15 minutes to prepare a test tube medium. Each of the 300 mutants that were subjected to colony isolation was inoculated on a test tube medium by means of an inoculating loop, and reciprocal shake culture (300 rpm) was carried out at 28° C. for 4 days. Subsequently, this culture product was centrifuged, and the resultant cells were analyzed for their carotenoid compound by high-performance liquid chromatography. As a result, one strain that exhibited a ratio of canthaxanthin at 60% by mass or higher relative to the total amount of carotenoid was obtained. The results of analysis of the carotenoid compound in this strain are shown in Table 5.

TABLE 5

| | Concentration of product | |
|---|---|---|
| | per culture solution | Ratio of product |
| Carotenoid compound | mg/L | % by mass |
| β-Carotene | 0.2 | 8.7 |
| Echinenone | 0.3 | 13.0 |
| 3-Hydroxyechinenone | 0.0 | 0.0 |
| Canthaxanthin | 1.5 | 65.2 |
| Adonirubin | 0.2 | 8.7 |
| β-Cryptoxanthin | 0.0 | 0.0 |
| Astaxanthin | 0.1 | 4.3 |
| Asteroidenone | 0.0 | 0.0 |
| Adonixanthin | 0.0 | 0.0 |
| Zeaxanthin | 0.0 | 0.0 |

All the publications, patents and patent applications cited herein are incorporated herein by reference in their entirety.

### INDUSTRIAL APPLICABILITY

The present invention provides a method for producing canthaxanthin, which is inexpensive, stably provided, and highly safe.

1. A method for producing canthaxanthin, comprising the steps of:
inducing a mutation in a parent astaxanthin-producing micro-organism strain, wherein the DNA sequence that corresponds to the micro-organism's 16S ribosomal RNA is substantially homologous to the sequence shown in SEQ ID NO: 1;
selecting a mutant that produces a higher mass percentage of canthaxanthin relative to that produced by the parent strain of the micro-organism; and
recovering canthaxanthin or a carotenoid mixture comprising canthaxanthin from a culture product of the selected mutant micro-organism.

2. The method of claim 1, wherein the mass percentage of canthaxanthin produced is at least 40% by mass relative to the total amount of carotenoid produced.

3. The method of claim 1, wherein each of the ratios of β-cryptoxanthin, zeaxanthin, 3-hydroxyechinenone, asteroidenone, adonirubin, adonixanthin, and astaxanthin produced from the selected mutant micro-organism is less than 20% by mass relative to the total amount of carotenoid produced.

4. The method of claim 1, wherein the astaxanthin-producing, micro-organism is selected from the group consisting of: E-396 strain known as (FERM BP-42833; a mutant, of the E-396 strain know as FERM BP-4283; the A-581-1 strain know as (FERM BP-4671); and a mutant of the A-581-1 strain known as FERM BP-4671.

5. The method of claim 2, wherein the astaxanthin-producing micro-organism is selected from the group consisting of: the E-396 strain known as FERM BP-4283; a mutant of the E-396 strain known as FERM BP-4283; the A-581-1 strain known as FERM BP-4671; and a mutant of the A-581-1 strain known as FERM BP-4671.

6. The method of claim 3, wherein the astaxanthin-producing micro-organism is selected from the group consisting of: the E-396 strain known as FERM BP-4283; a mutant of the E-396 strain known as FERM BP-4283; the A-581-1 strain known as FERM BP-4671; and a mutant of the A-581-1 strain known as FERM BP-4671.
